(19) United States
(12) Patent Application Publication
(10) Pub. No.: US 2017/0238862 A1
(43) Pub. Date: Aug. 24, 2017

(54) PHOTOACOUSTIC APPARATUS AND CONTROL METHOD FOR PHOTOACOUSTIC APPARATUS

(71) Applicant: CANON KABUSHIKI KAISHA, Tokyo (JP)

(72) Inventors: Kazuhiko Fukutani, Yokohama-shi (JP); Ryuichi Nanaumi, Tokyo (JP)

(21) Appl. No.: 15/503,425

(22) PCT Filed: Sep. 15, 2015
(86) PCT No.: PCT/JP15/04703

§ 371 Date: Feb. 13, 2017

(30) Foreign Application Priority Data
Sep. 22, 2014 (JP) 2014-192805

(51) Int. Cl. A61B 5/00

(52) U.S. Cl. CPC A61B 5/4312; A61B 5/0095; A61B 5/708

(57) ABSTRACT

a photoacoustic apparatus comprises a light source; a plurality of acoustic-wave receiving elements each configured to receive an acoustic wave generated from the object and convert into an electric signal; a supporting body configured to support the plurality of acoustic-wave receiving elements; a moving unit configured to move the supporting body; and an information acquiring unit configured to generate information in the object on the basis of the electric signal, wherein the photoacoustic apparatus performs measuring in first measurement positions and acquires a first electric signal, performs measuring in second measurement positions and acquires a second electric signal, and generates structural information and functional information in the object, and the first measurement positions and the second measurement positions are positions different from each other.

### TECHNICAL FIELD

The present invention relates to a photoacoustic apparatus that acquires information in an object.

### BACKGROUND ART

In the medical field, researches have been actively conducted for an optical imaging apparatus that irradiates, on a living body, light emitted from a light source such as a laser and images information in the living body on the basis of the incident light. As one kind of such an optical imaging technique, PhotoAcoustic Tomography (PAT) has been proposed in recent years.

When light such as pulse laser light is irradiated on a living body, which is an object, an acoustic wave (typically, an ultrasonic wave) is generated when the light is absorbed by a biological tissue in the object. This phenomenon is called photoacoustic effect. The acoustic wave generated by the photoacoustic effect is called photoacoustic wave. Tissues forming the object respectively have different absorption ratios of light energy. Therefore, sound pressures of photoacoustic waves generated by the tissues are also different. In the PAT, by receiving the generated photoacoustic wave with a probe and analyzing a reception signal, it is possible to image an initial sound pressure, a light absorption coefficient, and a distribution of light absorption energy density in the object.

In recent years, preclinical studies for imaging blood vessel structures and blood oxygen saturations of small animals using the photoacoustic tomography and clinical studies for applying the photoacoustic tomography to diagnosis of breast cancer, prostatic cancer, carotid artery plaque, and the like have been actively conducted.

### CITATION LIST

### Non Patent Literature

Non Patent Literature 1: “Dedicated 3D Photoacoustic Breast imaging”, Robert A. Kruger, Cherie M. Kuzmiak, Richard B. Lam, Daniel R. Reinecke, Stephen P. Del Rio, and Doreen Steed, Medical Physics 40, 113301(2013)

### SUMMARY OF INVENTION

### Technical Problem

Non Patent Literature 1 indicates that a clear image with reduced noise can be obtained by receiving photoacoustic waves in a plurality of positions while moving a supporting body on which a plurality of photoacoustic wave receiving elements are disposed in a hemispherical shape. The image is an image representing structure information in an object (e.g., a blood vessel structure in a breast).

Functional information in the object (a specific substance in the object; e.g., hemoglobin concentration included in blood or oxygen saturation in blood) can be obtained on the basis of structural information obtained by irradiating pulse lights having a plurality of wavelengths.

On the other hand, in a photoacoustic apparatus that acquires structural information and functional information in an object, further improvement of measurement accuracy is desired.

The present invention has been devised in view of such problems of the related art and it is an object of the present invention to improve measurement accuracy in a photoacoustic apparatus that acquires structural information and functional information in an object.

### Solution to Problem

The present invention in its one aspect provides a photoacoustic apparatus comprises a light source capable of irradiating pulse lights having a plurality of wavelengths on an object; a plurality of acoustic-wave receiving elements each configured to receive an acoustic wave generated from the object on which the pulse lights are irradiated and convert the acoustic wave into an electric signal; a supporting body configured to support the plurality of acoustic-wave receiving elements such that directional axes of at least a part of the plurality of acoustic-wave receiving elements are converged; a moving unit configured to move a position of the supporting body relatively to the object; and an information acquiring unit configured to generate information in the object on the basis of the electric signal, wherein when the supporting body is present in a plurality of measurement positions included in a first measurement position pattern, the light source performs irradiation of pulse light at a first wavelength and each of the acoustic-wave receiving elements converts the received acoustic wave into a first electric signal, when the supporting body is present in a plurality of measurement positions included in a second measurement position pattern, the light source performs irradiation of pulse light at a second wavelength and each of the acoustic-wave receiving elements converts the received acoustic wave into a second electric signal, the information acquiring unit generates structural information in the object on the basis of the first and second electric signals and generates functional information in the object on the basis of the first and second electric signals, and the measurement position included in the first measurement position pattern and the measurement position included in the second measurement position pattern are positions different from each other.

The present invention in its another aspect provides a control method for a photoacoustic apparatus including a light source capable of irradiating pulse lights having a plurality of wavelengths on an object, a plurality of acoustic-wave receiving elements each configured to receive an acoustic wave generated from the object on which the pulse lights are irradiated and convert the acoustic wave into an electric signal, and a supporting body configured to support the plurality of acoustic-wave receiving elements such that directional axes of at least a part of the plurality of acoustic-wave receiving elements are converged, the control method comprises a first measuring step of, while moving a position of the supporting body relatively to the object, performing irradiation of pulse light at a first wavelength and acquiring a first electric signal generated on the basis of acoustic waves received in a plurality of measurement positions included in a first measurement position pattern; a second measuring step of, while moving a position of the supporting body relatively to the object, performing irradiation of pulse light at a second wavelength and acquiring a second electric signal generated on the basis of acoustic waves received in a plurality of measurement positions included in a second measurement position pattern; a first information acquiring step of generating structural information in the object on the basis of the first and second electric signals; and a second information acquiring step of generating functional information in the object on the basis of the first and second electric signals, wherein the measurement position included in the first measurement position pattern and the measurement position included in the second measurement position pattern are positions different from each other.

The present invention in its another aspect provides a photoacoustic apparatus comprises a light source capable of irradiating pulse lights having a plurality of wavelengths on an object; a plurality of acoustic-wave receiving elements each configured to receive an acoustic wave generated from the object on which the pulse lights are irradiated and convert the acoustic wave into an electric signal; a supporting body configured to support the plurality of acoustic-wave receiving elements such that directional axes of at least a part of the plurality of acoustic-wave receiving elements are converged; a moving unit configured to move a position of the supporting body relatively to the object; a first control unit configured to, while moving the supporting body, perform irradiation of pulse light at a first wavelength and acquire a first electric signal generated on the basis of acoustic waves received in a plurality of measurement positions included in a first measurement position pattern; a second control unit configured to, while moving the supporting body, perform irradiation of pulse light at a second wavelength and acquire a second electric signal generated on the basis of acoustic waves received in a plurality of measurement positions included in a second measurement position pattern; a first information acquiring unit configured to generate structural information in the object on the basis of the first and second electric signals; and a second information acquiring unit configured to generate functional information in the object on the basis of the first and second electric signals, wherein the measurement position included in the first measurement position pattern and the measurement position included in the second measurement position pattern are positions different from each other.

### Advantageous Effects of Invention

According to the present invention, it is possible to improve measurement accuracy in a photoacoustic apparatus that acquires structural information and functional information in an object.

Further features of the present invention will become apparent from the following description of exemplary embodiments with reference to the attached drawings.

### BRIEF DESCRIPTION OF DRAWINGS

FIG. 1 is a system configuration diagram of a photoacoustic measurement apparatus according to a first embodiment.

FIG. 2 is a processing flowchart of the photoacoustic measurement apparatus according to the first embodiment.

FIGS. 3A to 3C are diagrams for explaining measurement position patterns in the first embodiment.

FIGS. 4A to 4E are diagrams for explaining effects in the first embodiment.

FIGS. 5A to 5C are diagrams for explaining measurement position patterns in a second embodiment.

### DESCRIPTION OF EMBODIMENTS

Embodiments of the present invention are explained in detail below with reference to the drawings. Note that numerical values, materials, shapes, arrangements, and the like used in the embodiments should be changed as appropriate according to the configurations and various conditions of apparatuses applied with the invention and do not limit the scope of the invention.

### First Embodiment

A photo acoustic apparatus according to a first embodiment is an apparatus that irradiates pulse light on an object and receives and analyzes a photoacoustic wave generated in the object because of the pulse light to visualize, i.e., image structural information and functional information in the object. The structural information means object information related to an initial sound pressure distribution and a light absorption energy density distribution or an absorption coefficient distribution derived from the initial sound pressure distribution and the light absorption energy density distribution and is mainly light absorption body structural information in the object, in particular, structural information of a blood vessel. The functional information is information calculated using photoacoustic signals and spectrum information acquired at a plurality of wavelengths and is mainly information concerning biological functions such as substance concentration in the object, in particular, concentration of oxygen and concentration of fat, collagen, and hemoglobin included in blood in a blood vessel. The photoacoustic apparatus according to the first embodiment can display the structural information and the functional information using images and numerical values.

### System Configuration

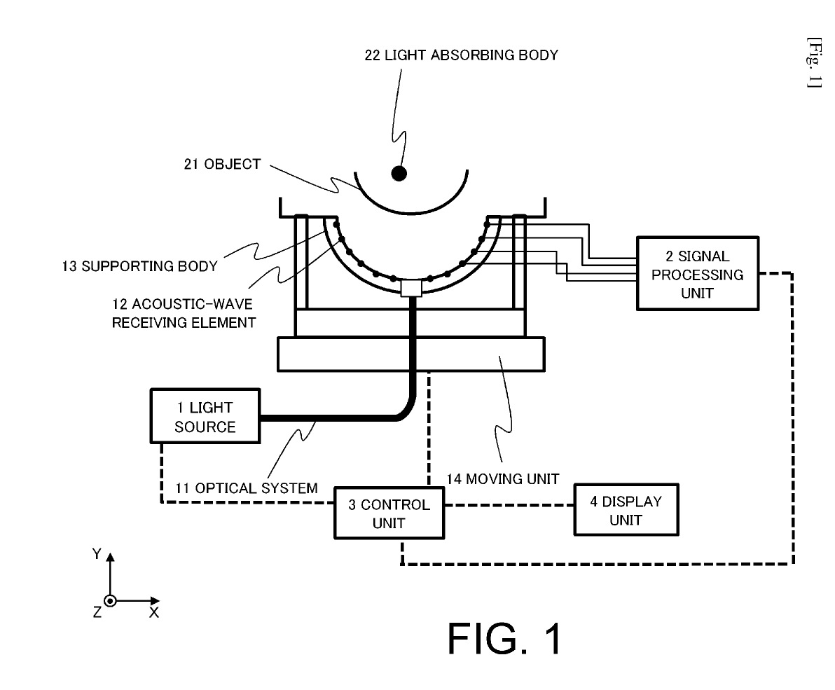

The configuration of the photoacoustic measurement apparatus according to this embodiment is explained with reference to FIG. 1. The photoacoustic measurement apparatus according to this embodiment includes a light source 1, an optical system 11, an acoustic-wave receiving element 12, a supporting body 13, a moving unit 14, a signal processing unit 2, a control unit 3, and a display unit 4.

In the following explanation, means configuring the photoacoustic measurement apparatus according to this embodiment are explained and an overview is explained concerning a method of measurement.

### Light Source 1

The light source 1 is means for generating pulse lights having a plurality of wavelengths and irradiating the pulse lights on an object. The light source is desirably a laser beam source in order to obtain a large output. However, a light emitting diode, a flash lamp, and the like can also be used instead of the laser. When the laser is used as the light source, various lasers such as a solid-state laser, a gas laser, a dye laser, and a semiconductor laser can be used.

Ideally, it is desirable to use an OPO laser, a dye laser, a Ti:sa laser, or an alexandrite laser of Nd:YAG excitation that have a strong output and can continuously change a wavelength. The light source may include a plurality of single-wavelength lasers having different wavelengths.

Timing, a waveform, intensity, and the like of the irradiation are controlled by the control unit 3. The control unit 3 that performs control of the light source 1 may be integrated with the light source.

The wavelength of the pulse light is desirably a specific wavelength absorbed by a specific component among components forming the object and is a wavelength at which light is propagated to the inside of the object. Specifically, when the object is a living body, the wavelength is desirably 500 nm or more and 1200 nm or less.

In order to effectively generate a photoacoustic wave, light has to be irradiated in a sufficiently short time according to a thermal characteristic of the object. When the object is a living body, pulse width of the pulse light generated from the light source is suitably approximately 10 nanoseconds to 50 nanoseconds.

Note that the light source 1 does not always need to be a part of the photoacoustic measurement apparatus according to this embodiment and may be connected outside.

### Optical System 11

The pulse light irradiated from the light source is irradiated toward the object through the optical system 11. The optical system 11 is configured by, for example, optical members such as a mirror that reflects light, a lens that magnifies the light, and a diffuser that diffuses the light. Besides, an optical fiber, a bundle optical fiber, an articulating arm in which a mirror is built in a lens barrel, and the like can also be used. As the optical system 11, any member may be used as long as the member can irradiate the pulse light emitted from the light source on the object in a desired shape. Note that it is more desirable to spread light to a certain degree of an area than condensing light with a lens from the viewpoint that a diagnosis region for the object can be expanded.

Note that, when a pulse light of a desired shape can be directly irradiated from the light source 1, the optical system 11 does not always need to be used.

### Object 21/Light Absorbing Body 22

Although an object 21 and a light absorbing body 22 do not configure the apparatus, the object 21 and the light absorbing body 22 are explained here. The photoacoustic measurement apparatus according to this embodiment has main purposes such as imaging of a blood vessel, diagnosis of malignant tumors, vascular diseases, and the like of a human and an animal, follow-up of chemical treatments, and the like. Therefore, as the object 21, a living body, specifically, a diagnosis target segment such as a breast, a finger, or a toe of a human or an animal is assumed. Note that, when the object is a small animal, not only a specific segment but also the entire small animal may be set as a target.

The light absorbing body 22 present on the inside of the object 21 is a segment having a relatively high absorption coefficient for light in the object. For example, when a measurement target is a human body, oxyhemoglobin or deoxyhemoglobin, a blood vessel including red blood cells, a malicious tumor including a newborn blood vessel, and the like correspond to the light absorbing body 22. Melanin and the like present on the surface of the object 21 also correspond to the light absorbing body 22. Note that the light absorbing body 22 maybe a dye such as methylene blue (MB) or indocyanine green, gold particulates, and a substance obtained by integrating or chemically moderating the dye such as the methylene blue (MB) or the indocyanine green, and the gold particulates.

### Acoustic-Wave Receiving Element 12

The acoustic-wave receiving element 12 is means for receiving an acoustic wave generated inside the object and converting the acoustic wave into an electric signal. The acoustic-wave receiving element is also called acoustic wave receiver and transducer. Note that the acoustic wave in the present invention is typically an ultrasonic wave and includes an elastic wave called sound wave, ultrasonic wave, photoacoustic wave, or optical ultrasonic wave.

The acoustic wave generated from a living body is an ultrasonic wave of 100 KHz to 100 MHz. Therefore, as the acoustic-wave receiving element 12, an acoustic element that can receive the frequency band is used. Specifically, a transducer that makes use of a piezoelectric phenomenon, a transducer that makes use of resonance of light, a transducer that makes use of a change in a capacity, and the like can be used. The acoustic-wave receiving element 12 is desirably an acoustic-wave receiving element having high sensitivity and a wide frequency band.

In this embodiment, a plurality of acoustic-wave receiving elements 12 are disposed. When the plurality of elements are used in this way, acoustic waves can be simultaneously received in a plurality of places. Therefore, it is possible to reduce a measurement time and reduce the influence of vibration and the like of the object.

### Supporting body 13

The supporting body 13 is a hemispherical member that supports the plurality of acoustic-wave receiving elements 12. FIG. 1 shows a sectional view of the supporting body 13 taken along an X-Y plane.

The supporting body 13 desirably has a shape for enabling the plurality of acoustic-wave receiving elements 12 to be disposed on closed curved surfaces surrounding the object 21. However, when it is difficult to dispose the plurality of acoustic-wave receiving elements 12 on all closed curved surfaces surrounding the object, as in this embodiment, the plurality of acoustic-wave receiving elements 12 may be disposed on the surface of the hemispherical supporting body 13 having an opening.

In general, an acoustic-wave receiving element has the highest reception sensitivity in the normal direction of a receiving surface (a surface). For example, when axes along the highest reception sensitivity direction (hereinafter referred to as directional axes) of the plurality of acoustic-wave receiving elements 12 are converged to the vicinity of a curvature center point of the hemispherical shape, a region where a highly accurate photoacoustic image is obtained is formed in the vicinity of the curvature center point. In particular, in this embodiment, the respective directional axes of the plurality of acoustic-wave receiving elements 12 are disposed to cross in the curvature center of the hemisphere. Consequently, resolution is increased in the region where the directional axes are converged. In this specification, the region having the high resolution in this way is referred to as high resolution region. Note that the high resolution region in this embodiment indicates a region from a point where resolution is the highest to a point where resolution is a half of the highest resolution.

Note that, the directional axes of the acoustic-wave receiving elements do not always have to cross as long as a desired high resolution region can be formed by converging the directional axes in a specific region.

In FIG. 1, an example is shown in which the acoustic-wave receiving elements are disposed using the hemispherical supporting body 13. However, the shape of the supporting body is not limited to this. The shape of the supporting body 13 may be any shape as long as the plurality of acoustic-wave receiving elements 12 can be disposed. For example, the supporting body 13 may have a curved surface different from the illustrated curved surface or may have a flat surface.

Note that it is desirable that a member (e.g., water) for matching acoustic impedance can be interposed between the supporting body 13 and the object 21.

### Moving Unit 14

The moving unit 14 is means for moving the position of the supporting body 13 relatively to the object 21. The plurality of acoustic-wave receiving elements 12 disposed on the supporting body 13 can be moved relatively to the object 21 by the moving unit 14. Therefore, it is possible to receive photoacoustic waves in a plurality of positions. A specific moving method may be a step-and-repeat method for repeating movement and rest or may be continuous movement. However, the continuous movement is more desirable because an overall moving time is short and a burden on the object can be reduced and a change in acceleration is small and the influence of a swing of the apparatus can be reduced.

Note that the moving unit 14 is desirably configured to move the supporting body 13 and the optical system 11 (at least an emission port of pulse light) in synchronization with each other. Consequently, since a relation between a receiving position of an acoustic wave and an irradiation position of light is kept fixed, it is possible to acquire more uniform object information.

In order to increase an amount of light propagating to the inside of the object 21, it is desirable to increase an irradiation area. However, the irradiation area is limited from the viewpoint of, for example, costs of the light source. When the object is a human body, an irradiation area of pulse light is limited by the American National Standards Institute (ANSI) standard as well.

Since the acoustic-wave receiving element has directivity, efficiency of use of a light amount is low even if light is irradiated on a region with low reception sensitivity. That is, it is inefficient to irradiate light on the entire object. Therefore, in this embodiment, the optical system 11 is moved in synchronization with the supporting body 13 and light is irradiated only on a region with high sensitivity.

In this embodiment, the emission port of pulse light is disposed in the center (a pole portion) of the supporting body 13. The pulse light is irradiated toward the curvature center of the supporting body 13. Consequently, light is always irradiated on regions with high sensitivity of the plurality of acoustic-wave receiving elements 12. Since the supporting body 13 and the optical system 11 are integrated, it is possible to move the high resolution region while keeping the relation between the measurement position of a photoacoustic wave and the irradiation position of light.

The pulse light is irradiated at a plurality of timings while the moving unit 14 moves the supporting body 13. Consequently, it is possible to dispose the high resolution region in a different position at every measurement timing. It is possible to expand the region with high resolution. Note that, in order to reduce fluctuation in resolution in a region to be imaged, the moving unit 14 more desirably move the supporting body 13 such that a plurality of high resolution regions overlap.

### Signal Processing Unit 2

The signal processing unit 2 is means for amplifying an electric signal (hereinafter, photoacoustic signal) obtained by the acoustic-wave receiving element 12 and converting the photoacoustic signal into a digital signal. Typically, the signal processing unit 2 is configured by an amplifier, an A/D converter, an FPGA (Field Programmable Gate Array) chip, and the like. When a plurality of signals are obtained from the acoustic-wave receiving element 12, it is desirable that the signal processing unit 2 can simultaneously process the plurality of signals. Note that the photoacoustic signal in this specification is a concept including both of an analog electric signal obtained by the acoustic-wave receiving element 12 and a digital signal converted by the signal processing unit 2.

The signal processing unit 2 may integrates photoacoustic signals received in the same position on the object into one signal. A method of integration may be a method of adding up the signals or may be a method of averaging the signals. The method of integration may be a method of weighting and adding up the signals.

### Control Unit 3

The control unit 3 is means for controlling the components included in the photoacoustic measurement apparatus. Specifically, the control unit 3 controls the intensity of light irradiated on the object, irradiation timing of the light, reception timing of an acoustic wave, the position of the moving unit, and the like.

The control unit 3 is also means (an information acquiring unit) for processing a photoacoustic signal output by the signal processing unit 2 and generating an image. As a method of generating an image on the basis of a photoacoustic signal, there are, for example, a Fourier transform method, a universal back projection method, a filtered back projection method, and an iterative reconstruction method. However, any method may be used.

In this embodiment, the control unit 3 is a computer. Typically, the control unit 3 is configured from a device such as a CPU, a GPU, or an A/D converter and a circuit such as an FPGA or an ASIC. Note that the control unit 3 may be configured from a plurality of devices and circuits.

The control unit 3 includes, as a storing unit, a storage medium such as a ROM, a RAM, or a hard disk. The storage medium may be configured from a plurality of storage media.

The control unit 3 is desirably configured to be capable of simultaneously subjecting a plurality of signals to pipeline processing. Consequently, it is possible to reduce time until object information is acquired.

Note that a computer program for causing the control unit 3 to execute the respective kinds of processing can be stored in a non transitory recording medium included in the storing unit.

### Display Unit 4

The display unit 4 is a device that displays an image and a numerical value representing structural information or functional information generated by the control unit 3. Typically, a liquid crystal display or the like is used. However, the display unit 4 maybe a display of another system such as a plasma display, an organic EL display, or an FED. That is, the control unit 3 causes the display unit 4 to display the structural information or the functional information.

Note that the display unit 4 does not always need to be a part of the photoacoustic measurement apparatus according to this embodiment and may be connected outside.

### Measuring Method for an Object

A method of measuring a living body (in particular, a breast), which is an object, with the photoacoustic measurement apparatus according to this embodiment is explained.

First, a pulse light having a specific wavelength emitted from the light source 1 is irradiated on the object. The pulse light is led to the surface of the object and irradiated on the surface while being processed into a desired shape by the optical system 11 including, for example, a lens, a mirror, an optical fiber, and a diffuser. When a part of energy of light propagated on the inside of the object 21 is absorbed by the light absorbing body 22 such as a blood vessel, a photoacoustic wave (typically, an ultrasonic wave) is generated by thermal expansion of the light absorbing body 22.

The generated acoustic wave is propagated inside the object, received by the plurality of acoustic-wave receiving elements 12 disposed on the supporting body 13, and converted into an analog electric signal.

The photoacoustic measurement apparatus according to this embodiment performs measurement at a plurality of timings while the moving unit 14 moves the supporting body 13.

Note that “measurement” in this specification indicates irradiating pulse light and receiving a photoacoustic wave generated by the pulse light. “Measurement position” indicates the position of the acoustic-wave receiving element at the time when the pulse light is irradiated, more specifically, a center position of the supporting body 13 with respect to the object.

In this specification, a set of coordinates of measurement positions where measurement is performed at the plurality of timings using the pulse light having the specific wavelength is referred to as “measurement position pattern”.

Photoacoustic signals output from the plurality of acoustic-wave receiving elements 12 are subjected to amplification processing and digital conversion processing in the signal processing unit 2 and thereafter stored in the storing unit in the control unit 3. That is, photoacoustic signals output from the plurality of acoustic-wave receiving elements 12 at different measurement timings are stored in the storing unit in the control unit 3.

In this embodiment, the photoacoustic measurement apparatus performs measurement using a plurality of measurement position patterns while switching the wavelength of pulse light. That is, in the control unit 3, a set of photoacoustic signals obtained by the measurement is stored for each measurement position pattern (for each wavelength).

An example of the measurement position patterns and a specific measurement method are explained below.

Note that propagation speed of a photoacoustic wave is high compared with speed of movement of the supporting body 13 by the moving unit 14. Therefore, the acoustic-wave receiving element 12 receives the photoacoustic wave substantially simultaneously with timing when the pulse light is irradiated. Therefore, a movement amount of the supporting body 13 from the irradiation of the pulse light on the object until reception of the photoacoustic wave by the plurality of acoustic-wave receiving elements 12 can be neglected. In this embodiment, timing when the pulse light is irradiated is timing when the photoacoustic wave is received (hereinafter, measurement timing). Note that, since the supporting body 13 supports the plurality of acoustic-wave receiving elements 12, by specifying the position of the supporting body 13, it is possible to specify the positions of the plurality of acoustic-wave receiving elements 12.

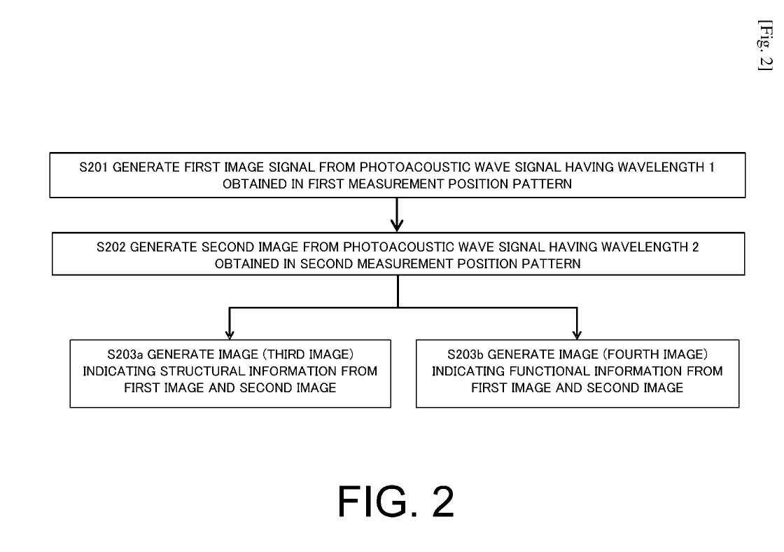

Subsequently, the control unit 3 acquires, using the stored photoacoustic signals, object information in a region of interest (ROI) designated by the user. FIG. 2 is a flowchart for explaining a flow of acquisition of the object information.

The object information is represented by voxels when three-dimensional information is acquired and represented by pixels when two-dimensional information is acquired. As a method of acquiring object information from the photoacoustic signals, publicly-known reconstruction methods such as Universal Back Projection (UBP) and Filtered Back Projection (FBP) can be used. According to these reconstruction methods, it is possible to acquire an image representing object information from a plurality of photoacoustic signals corresponding to a certain measurement position patterns (S201).

It is possible to acquire an image corresponding to different wavelengths by performing the same processing using a plurality of photoacoustic signals acquired using different measurement position patterns while irradiating pulse lights having different wavelengths (S202). Both of the images acquired in steps S201 and S202 are images representing an absorption coefficient (i.e., structural information) in the object.

In this specification, the image acquired in step S201 is referred to as first image and the image acquired in step S202 is referred to as second image.

It is possible to generate an image representing an absorption coefficient in the object, i.e., an image with less noise using the first image and the second image (S203a). In this specification, an image acquired in step S203a is referred to as third image.

The first image and the second image are images generated on the basis of the pulse lights having different wavelengths. Therefore, it is possible to generate, using the two images, an image representing spectrum information (functional information; e.g., oxygen saturation) in the object (S203b).

In this specification, the image acquired in step S203b is referred to as fourth image.

The generated images (e.g., the third image and the fourth image) are output to the display unit 4 and served for diagnosis or the like.

### Measurement Position Pattern

A specific example of a measurement position pattern and an image obtained by performing measurement using the measurement position pattern are explained. As explained above, the photoacoustic measurement apparatus according to this embodiment performs measurement for each wavelength of pulse light using a plurality of different measurement position patterns.

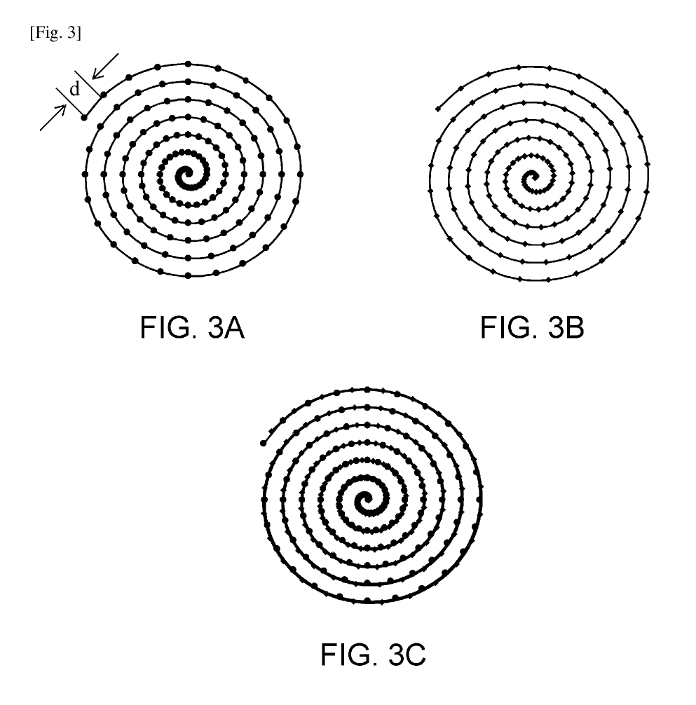

FIG. 3A is a diagram in which the center position of the supporting body 13 is plotted at every measurement timing when measurement is performed using a certain wavelength (hereinafter, first wavelength). That is, FIG. 3A is an example of a measurement position pattern corresponding to the first waveform. The measurement position pattern is referred to as first measurement position pattern. In this embodiment, the control unit 3 controls a moving route and measurement timing of the supporting body 13 such that photoacoustic waves can be acquired in measurement positions indicated by black circles in FIG. 3A in the measurement performed using the first wavelength.

On the other hand, FIG. 3B is a diagram in which the center position of the supporting body 13 is plotted at every measurement timing when measurement is performed using a wavelength (a second wavelength) different from the first wavelength in the photoacoustic measurement apparatus according to this embodiment. That is, FIG. 3B is an example of a measurement position pattern corresponding to the second wavelength. The measurement position pattern is referred to as second measurement position pattern. The control unit 3 controls a moving route and measurement timing of the supporting body 13 such that photoacoustic waves can be acquired in measurement positions indicated by black circles in FIG. 3B in the measurement performed using the second wavelength.

The first measurement position pattern and the second measurement position pattern are compared. FIG. 3C is a superimposed pattern of both the measurement position patterns. Tracks of the first measurement pattern and the second measurement position pattern are substantially the same. However, measurement positions included in the patterns are different. Therefore, as shown in FIG. 3C, the measurement positions in the patterns do not overlap.

In this embodiment, the control unit 3 sets a moving route and measurement timing of the supporting body 13 such that a distribution of the measurement positions by the first wavelength is the first measurement position pattern and a distribution of the measurement positions by the second wavelength is the second measurement position patterns. Specifically, when the center of the supporting body 13 is present in the measurement positions included in the first measurement position pattern, the control unit 3 generates pulse light at the first wavelength and performs measurement. When the center of the supporting body 13 is present in the measurement positions included in the second measurement position pattern, the control unit 3 generates pulse light at the second wavelength and performs measurement.

The control unit 3 generates the first image on the basis of a photoacoustic signal corresponding to the first measurement position pattern and generates the second image on the basis of a photoacoustic signal corresponding to the second measurement position pattern.

A relation between the first measurement position pattern and the second measurement position pattern is as explained below. That is, when a maximum distance between adjacent measurement positions included in the first measurement pattern is represented as d, a measurement position included in the first measurement position pattern and a measurement position included in the second measurement position pattern and corresponding to the measurement position is present at a distance within d.

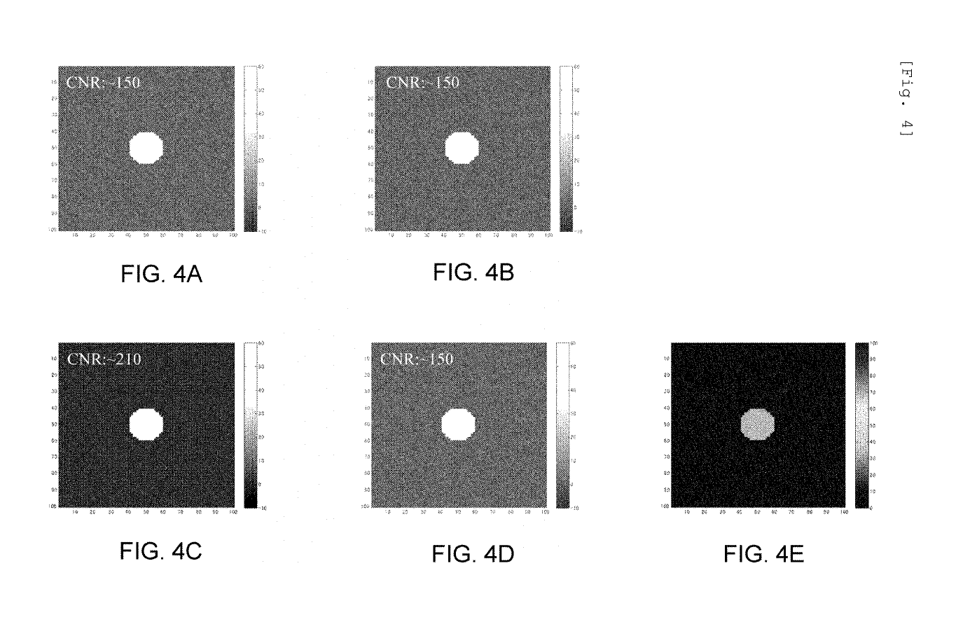

Effects obtained when a measurement position is shifted for each wavelength of pulse light are explained below. FIG. 4A is an example of an image (a first image) reconstructed by performing measurement using the first measurement position pattern at the first wavelength. The image is an image representing an absorption coefficient distribution in the object. A portion with high brightness represents the position of the light absorbing body. As it is seen from the figure, a spherical light absorbing body is present in the center part of the image. Image noise due to reconstruction artifact is seen in parts other than the image center part.

FIG. 4B is an example of an image (a second image) reconstructed by performing measurement using the second measurement position pattern at the second wavelength. In this figure as well, a portion with high brightness represents the position of the light absorbing body. These images are images indicating the position and the shape of the light absorbing body, that is, images indicating structural information in the object.

In FIG. 4A and FIG. 4B, when a contrast noise ratio (CNR), which is a ratio of an average of pixel values corresponding to the light absorbing body and a square average of pixel values in the other portions, was calculated, the CNR was approximately 150 in both the images.

FIG. 4C is an image (a third image) obtained by averaging the first image and the second image. In this figure as well, a portion with high brightness represents the position of the light absorbing body. The CNR of this image is approximately 210. The CNR greatly increased compared with FIG. 4A and FIG. 4B.

On the other hand, FIG. 4D is an image obtained by performing measurement using the same measurement position pattern (e.g., FIG. 3A) and using the first wavelength and the second wavelength and averaging obtained two images. The CNR of the image was approximately 150.

In general, noise in an image generated by the photoacoustic measurement apparatus can be classified into image reconstruction artifact and system noise. Both of these kinds of noise are reduced as measurement positions increase. The image shown in FIG. 4C is mathematically equivalent to an image generated on the basis of a photoacoustic signal obtained in the measurement position pattern shown in FIG. 3C. That is, by changing the measurement position pattern for each wavelength to perform measurement and averaging images, it is possible to obtain an effect same as an effect obtained by doubling the measurement positions. It is possible to improve the image quality of an image representing structural information in the object.

On the other hand, the image shown in FIG. 4D is equivalent to an image generated on the basis of photoacoustic signals acquired by performing measurement twice using the first measurement position pattern. When acoustic waves are received a plurality of times in the same measurement position, image reconstruction artifact is not reduced and only noise due to system noise is reduced. For example, when a main cause of noise that occurs in the images shown in FIG. 4A and FIG. 4B is the system noise, the CNR in FIG. 4C is improved compared with FIG. 4A and 4B but is the same compared with FIG. 4D.

From the above, it is seen that more highly accurate structural information can be acquired by acquiring photoacoustic waves using different measurement position patterns and generating, on the basis of respective kinds of obtained information, an image indicating structural information in the object.

The image representing the structural information in the object is explained above. Next, an image representing functional information in the object is explained.

In this embodiment, as the image representing the functional information in the object, an image representing concentration of a substance forming the light absorbing body is generated. Assuming that the light absorbing body is formed of only hemoglobin and oxygenated hemoglobin, an image representing oxygen saturation in the light absorbing body is generated.

Since the oxygen saturation is spectral information, the oxygen saturation needs to be calculated using an absorption coefficient distribution acquired using different wavelengths. FIG. 4E is an image (a fourth image) representing oxygen saturation in the light absorbing body calculated from the first image shown in FIG. 4A and the second image shown in FIG. 4B. The fourth image can be calculated by a known method.

As explained above, the photoacoustic measurement apparatus according to the first embodiment performs a plurality of times of measurement while changing the measurement position pattern and generates an image representing structural information and an image representing functional information in the object on the basis of respective obtained images.

When pulse lights having different wavelengths are irradiated to calculate functional information, if measurement positions increase, calculation accuracy is deteriorated. Therefore, in the present invention, it is important that, as explained above, the respective corresponding measurement positions in the patterns are within the distance d. By performing measurement using such measurement position patterns, it is possible to reduce reconstruction artifact that occurs in the structural information while maintaining accuracy of the functional information. That is, it is possible to attain both of the accuracy of the structural information and the accuracy of the functional information.

### Second Embodiment

In the first embodiment, measurement for the object is performed using the spiral measurement position pattern. On the other hand, a second embodiment is an embodiment in which measurement is performed using a linear measurement position pattern.

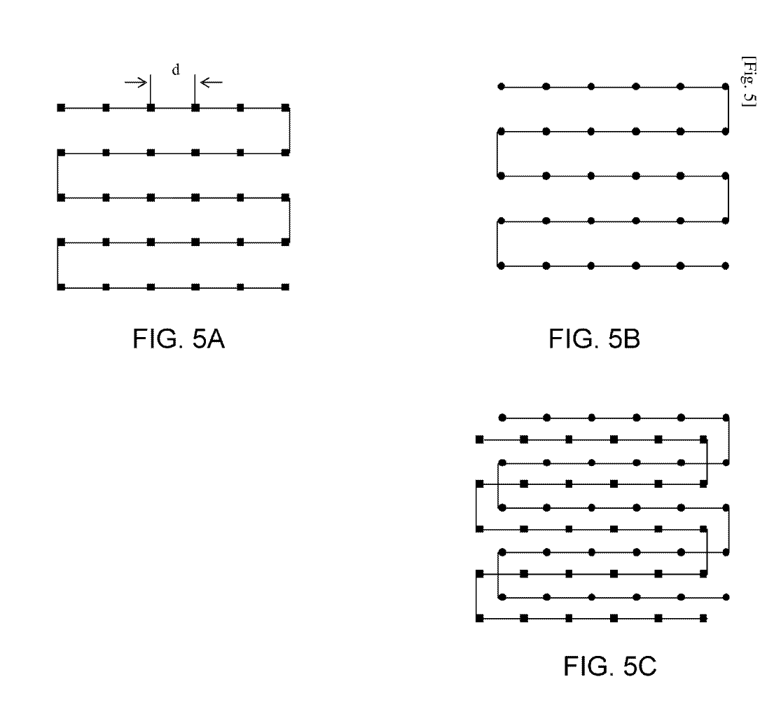

FIGS. 5A to 5C are diagrams showing examples of measurement position patterns in this embodiment. In the second embodiment, the control unit 3 controls a moving route of the supporting body 13 and measurement timing such that a first measurement position pattern is a pattern shown in FIG. 5A and a second measurement position pattern is a pattern shown in FIG. 5B.

A relation same as the relation in the first embodiment is present between the first measurement position pattern and the second measurement position pattern. That is, when a maximum distance between adjacent measurement positions included in the first measurement pattern is represented as d, a measurement position included in the first measurement position pattern and a measurement position included in the second measurement position pattern and corresponding to the measurement position is present at a distance within d.

FIG. 5C is a pattern obtained by superimposing the first measurement position pattern and the second measurement position pattern. As it is seen from the figure, when the two patterns are compared, the measurement positions are matched by translation of the distance d or less.

In the second embodiment, as in the first embodiment, it is possible to obtain an effect of reducing reconstruction artifact, which occurs in structural information, while maintaining accuracy of functional information.

### Example 1

A specific example of the photoacoustic measurement apparatus applied with the first embodiment is explained.

In the photoacoustic measurement apparatus according to an example 1, as the light source 1, a Ti:sa laser system of YAG laser excitation with a double wave was used. The Ti:sa laser can irradiate light having a wavelength between 700 nm and 900 nm on the object. Note that a laser beam is set to be irradiated on the surface of the object 21 after being expanded to a radius of approximately 1 cm using the optical system 11 such as a mirror and a beam expander.

As the acoustic-wave receiving elements 12, 512 piezoelectric elements are disposed in a spiral shape on the hemispherical supporting body 13. The hemispherical supporting body 13 is configured to be movable with respect to the object by the moving unit 14 that moves in an XY direction.

The signal processing unit 2 has a function of simultaneously receiving signals output from the 512 acoustic-wave receiving elements and, after applying amplification and digital conversion to the signals, transferring the signals to the control unit 3. The signal processing unit 2 sets timing of light irradiation as reception start timing and performs acquisition of a photoacoustic signal at a sampling frequency of 20 MHz.

The object 21 is a hemispherical phantom simulating a living body and is formed of titanium oxide functioning as a scattering body and urethane rubber mixed with two kinds of ink simulating and absorption spectra of blood functioning as an absorbing body. Spherical black rubber having a diameter of 0.5 mm is embedded in the center of the hemispherical urethane phantom as the light absorbing body 22. The size of the phantom is a diameter of 80 mm. The urethane phantom is fixed by a transparent plastic cup and is in contact with the acoustic-wave receiving element 12 via water functioning as an acoustic matching member.

In the example 1, lights having wavelengths of 756 nm and 797 nm were alternately irradiated at 10 Hz on the phantom using two Ti:sa lasers to respectively scan the supporting body 13 such that measurement position patterns were the measurement position patterns shown in FIG. 3A and FIG. 3B. Specifically, the supporting body 13 was scanned such that a measurement position pattern corresponding to the wavelength 756 nm was the measurement position pattern shown in FIG. 3A and a measurement position pattern corresponding to the wavelength 797 nm was the measurement position pattern shown in FIG. 3B. A plurality of photoacoustic signals respectively obtained in the measurement position patterns were stored in the control unit 3.

A first image was generated by the back projection method using the photoacoustic signals obtained by irradiating pulse light having the wavelength of 756 nm. The obtained image is an image representing an initial sound pressure distribution in the object.

A second image was generated by the back projection method using the photoacoustic signals obtained by irradiating pulse light having the wavelength of 797 nm. The obtained image is also an image representing the initial sound pressure distribution in the object.

Subsequently, a weighted average corresponding to a light amount was calculated for the first image and the second image to generate a third image. The third image is an image indicating structural information of the light absorbing body. When CNRs of the third image and the first and second images were calculated, a result indicating that the CNR of the third image increased by approximately √2 times was obtained.

Subsequently, an image representing a concentration ratio of two kinds of ink was generated using a ratio of the first image and the second image and absorption spectra of the two kinds of ink. The generated image is a fourth image indicating functional information of the light absorbing body. As a result, a concentration ratio substantially the same as the concentration ratio of the ink forming the light absorbing body was calculated.

### Example 2

An example 2 is a specific example of the photoacoustic measurement apparatus applied with the second embodiment. The apparatus configuration of the photoacoustic measurement apparatus according to the example 2 is the same as the apparatus configuration in the example 1 except points explained below. A phantom used in the example 2 is the same as the phantom in the example 1.

In the example 2, lights having wavelengths of 755 nm and 797 nm were alternately irradiated using an alexandrite laser to respectively scan the supporting body 13 such that measurement position patterns were the measurement position patterns shown in FIG. 5A and FIG. 5B. Specifically, the supporting body 13 was scanned such that a measurement position pattern corresponding to the wavelength 755 nm was the measurement position pattern shown in FIG. 5A and a measurement position pattern corresponding to the wavelength 797 nm was the measurement position pattern shown in FIG. 5B. A plurality of photoacoustic signals respectively obtained in the measurement position patterns were stored in the control unit 3.

An image was generated using a model base image reconstruction method using the photoacoustic signals obtained by irradiating pulse light having the wavelength of 755 nm. The obtained image is an image representing an initial sound pressure distribution in the object. Subsequently, light amount distributions in the respective light irradiations were calculated using the shape of the phantom, light irradiation patterns, irradiated light amounts in the light irradiations, and an optical coefficient of the phantom. An image (a first image) indicating an absorption coefficient distribution in the object was generated from the light amount distribution and an image, which is an initial sound pressure distribution.

Subsequently, an image was generated using the model base image reconstruction method using the photoacoustic image obtained by irradiating pulse light having the wavelength of 797 nm. Thereafter, an image (a second image) indicating an absorption coefficient distribution in the object was generated by the same method.

Subsequently, a third image was generated by averaging the first image and the second image. When CNRs of the third image and the first and second images were calculated, a result indicating that the CNR of the third image increased by approximately √2 times was obtained.

Subsequently, an image representing a concentration ratio of two kinds of ink was generated using a ratio of the first image and the second image and absorption spectra of the two kinds of ink. The generated image is a fourth image indicating functional information of the light absorbing body. As a result, a concentration ratio substantially the same as the concentration ratio of the ink forming the light absorbing body was calculated.

### Example 3

A example 3 is an example in which wavelengths of pulse lights used for measurement are four wavelengths. The apparatus configuration of the photoacoustic measurement apparatus according to the example 3 is the same as the apparatus configuration in the example 1 except that the light source 1 can selectively emit the pulse lights having the four wavelengths. A phantom used in the example 3 is the same as the phantom in the example 1.

In the example 3, first, light having a wavelength of 700 nm was irradiated on the phantom from the Ti:sa laser to scan the supporting body 13 such that a measurement position pattern was the measurement position pattern shown in FIG. 3A. An obtained time-series photoacoustic signal was stored in the control unit 3.

Subsequently, measurement was performed in the same manner using lights having wavelengths of 730 nm, 756 nm, and 797 nm. As measurement position patterns used at the wavelengths, patterns obtained by respectively tilting the pattern shown in FIG. 3A by 5 degrees, 10 degrees, and 15 degrees from a scanning center were used. Time-series photoacoustic signals obtained in the measurement position patterns were stored in the control unit 3.

Subsequently, images corresponding to the wavelengths were generated by the back projection method using the photoacoustic signals corresponding to the wavelengths. Obtained four images are images representing an initial sound pressure distribution in the object.

Subsequently, the four images corresponding to the wavelengths were averaged to generate a third image indicating structural information of the light absorbing body. Note that a CNR of this image was higher than CNRs of the images at the wavelengths by approximately two times.

Subsequently, as in the first embodiment, an image representing a concentration ratio of two kinds of ink was generated on the basis of the images corresponding to the wavelengths. The generated image is a fourth image indicating functional information of the light absorbing body. As a result, a concentration ratio substantially the same as the concentration ratio of the ink forming the light absorbing body was calculated.

As explained above, according to the examples 1 to 3, it was found that, compared with the conventional example, information can be obtained more accurately in the photoacoustic measurement apparatus that acquires photoacoustic signals using a plurality of wavelength and generates a structural information image and a functional information image of the inside of the object.

### Modification

Note that the embodiments (the examples) are illustrations in explaining the present invention. The present invention can be changed or combined as appropriate to be carried out in a range not departing from the gist of the invention.

For example, the present invention can also be carried out as a photoacoustic apparatus including at least a part of the processing explained above. The present invention can also be carried out as a control method for the photoacoustic apparatus including at least a part of the processing. The processing and the means explained above can be freely combined to be carried out unless technical contradiction occurs.

In the embodiments (the examples), the measurement using the first wavelength and the measurement using the second wavelength are performed in order. However, a wavelength may be switched every time pulse light is irradiated. If there is a light source or a plurality of light sources that can switch a wavelength of light in time shorter than an irradiation interval of the pulse light, such a form can be adopted. In this case, after sequentially performing, in the measurement points, measurement at wavelengths corresponding to the measurement points, an image only has to be reconstructed for each of the wavelengths on the basis of acquired photoacoustic signals.

In the embodiments (the examples), the wavelengths of the pulse light used for the measurement are two wavelengths and four wavelengths. However, the number of wavelengths may be other numbers. If the measurement positions included in the measurement position pattern are different for each of the wavelengths, it is possible to obtain the effects of the invention.

In the embodiments (the examples), the pattern obtained by rotating or translating the first measurement position pattern is the second measurement position pattern. However, the measurement position pattern is not limited to these patterns. For example, only the measurement positions included in the measurement patterns may be moved while keeping the measurement position pattern.

In the embodiments (the examples), the apparatus that outputs the information in the object as the image is explained. However, the information in the object may be output a form other than the image such as a numerical value.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-192805, filed on Sep. 22, 2014, which is hereby incorporated by reference herein in its entirety.

### REFERENCE SIGNS LIST

1 Light source
2 Signal processing unit
3 Control unit
12 Acoustic-wave receiving element
13 Supporting body
14 Moving unit

1. A photoacoustic apparatus comprising:
a light source capable of irradiating pulse lights having a plurality of wavelengths on an object;
a plurality of acoustic-wave receiving elements each configured to receive an acoustic wave generated from the object on which the pulse lights are irradiated and convert the acoustic wave into an electric signal;
a supporting body configured to support the plurality of acoustic-wave receiving elements such that directional axes of at least a part of the plurality of acoustic-wave receiving elements are converged;
a moving unit configured to move a position of the supporting body relatively to the object; and
an information acquiring unit configured to generate information in the object on the basis of the electric signal, wherein
when the supporting body is present in a plurality of measurement positions included in a first measurement position pattern, the light source performs irradiation of pulse light at a first wavelength and each of the acoustic-wave receiving elements converts the received acoustic wave into a first electric signal,
when the supporting body is present in a plurality of measurement positions included in a second measurement position pattern, the light source performs irradiation of pulse light at a second wavelength and each of the acoustic-wave receiving elements converts the received acoustic wave into a second electric signal,
the information acquiring unit generates structural information in the object on the basis of the first and second electric signals and generates functional information in the object on the basis of the first and second electric signals, and
the measurement position included in the first measurement position pattern and the measurement position included in the second measurement position pattern are positions different from each other.

2. The photoacoustic apparatus according to claim 1, wherein when a maximum distance between adjacent measurement positions included in the first measurement position pattern is represented as d, a distance between the measurement position included in the first measurement position pattern and the measurement position included in the second measurement position pattern is within d.

3. The photoacoustic apparatus according to claim 2, wherein the second measurement position pattern is a pattern obtained by rotating the first measurement position pattern.

4. The photoacoustic apparatus according to claim 2, wherein the second measurement position pattern is a pattern obtained by translating the first measurement position pattern.

5. The photoacoustic apparatus according to claim 1, wherein the information acquiring unit generates an image representing the structural information and an image representing the functional information and outputs the images to a display device.

6. A control method for a photoacoustic apparatus including a light source capable of irradiating pulse lights having a plurality of wavelengths on an object, a plurality of acoustic-wave receiving elements each configured to receive an acoustic wave generated from the object on which the pulse lights are irradiated and convert the acoustic wave into an electric signal, and a supporting body configured to support the plurality of acoustic-wave receiving elements such that directional axes of at least a part of the plurality of acoustic-wave receiving elements are converged, the control method comprising:
a first measuring step of, while moving a position of the supporting body relatively to the object, performing irradiation of pulse light at a first wavelength and acquiring a first electric signal generated on the basis of acoustic waves received in a plurality of measurement positions included in a first measurement position pattern;
a second measuring step of, while moving a position of the supporting body relatively to the object, performing irradiation of pulse light at a second wavelength and acquiring a second electric signal generated on the basis of acoustic waves received in a plurality of measurement positions included in a second measurement position pattern;
a first information acquiring step of generating structural information in the object on the basis of the first and second electric signals; and
a second information acquiring step of generating functional information in the object on the basis of the first and second electric signals,
wherein the measurement position included in the first measurement position pattern and the measurement position included in the second measurement position pattern are positions different from each other.

7. The control method for a photoacoustic apparatus according to claim 6, wherein when a maximum distance between adjacent measurement positions included in the first measurement position pattern is represented as d, a distance between the measurement position included in the first measurement position pattern and the measurement position included in the second measurement position pattern is within d.

8. A photoacoustic apparatus comprising:
a light source capable of irradiating pulse lights having a plurality of wavelengths on an object;
a plurality of acoustic-wave receiving elements each configured to receive an acoustic wave generated from the object on which the pulse lights are irradiated and convert the acoustic wave into an electric signal;
a supporting body configured to support the plurality of acoustic-wave receiving elements such that directional axes of at least a part of the plurality of acoustic-wave receiving elements are converged;
a moving unit configured to move a position of the supporting body relatively to the object;
a first control unit configured to, while the supporting body is being moved, perform irradiation of pulse light at a first wavelength and acquire a first electric signal generated on the basis of acoustic waves received in a plurality of measurement positions included in a first measurement position pattern;
a second control unit configured to, while the supporting body is being moved, perform irradiation of pulse light at a second wavelength and acquire a second electric signal generated on the basis of acoustic waves received in a plurality of measurement positions included in a second measurement position pattern;
a first information acquiring unit configured to generate structural information in the object on the basis of the first and second electric signals; and
a second information acquiring unit configured to generate functional information in the object on the basis of the first and second electric signals,
wherein the measurement position included in the first measurement position pattern and the measurement position included in the second measurement position pattern are positions different from each other.
